(19) United States
(12) Patent Application Publication
(10) Pub. No.: US 2023/0230189 A1
(43) Pub. Date: Jul. 20, 2023

(54) AN INTEGRATED END TO END SYSTEM AND METHOD FOR ACCIDENT AND MEDICAL EMERGENCY RESPONSE

(71) Applicant: PRAVEEN CHOUDHARY, JAIPUR (IN)

(72) Inventor: PRAVEEN CHOUDHARY, JAIPUR (IN)

(21) Appl. No.: 17/998,260

(22) PCT Filed: Jun. 29, 2020
(86) PCT No.: PCT/IB2020/056113

§ 371 Date: Nov. 9, 2022

(30) Foreign Application Priority Data
May 13, 2020 (IN) 202011020143

(51) Int. Cl. G06Q 50/26; G16H 40/20

(52) U.S. Cl. CPC G06Q 50/265; G16H 40/20; G06Q 20/34

(57) ABSTRACT

An integrated end to end system and method for accident and medical emergency response are disclosed. The system includes a registration subsystem configured to register one or more entities, an emergency determining subsystem configured to determine one or more emergencies, a healthcare service selection subsystem configured to enable the one or more affected users to select one or more healthcare, a healthcare service provider connecting subsystem configured to connect the one or more healthcare service providers to the one or more affected users, a referral subsystem configured to refer the one or more affected users from one or more rural areas (a primary health centre) to a secondary health centre and from the secondary health centre to a tertiary health centre, a payment receiving subsystem configured to receive a payment from the one or more affected users via the payment card shared within a family as a family tree profile.

This International Application claims priority from a complete patent application filed in India having patent application number 202011020143, filed on May 13, 2020 and titled “AN INTEGRATED END TO END SYSTEM AND METHOD FOR ACCIDENT AND MEDICAL EMERGENCY RESPONSE”.

### FIELD OF INVENTION

Embodiments of the present disclosure relate to health care services, and more particularly to, an integrated end to end system and method for accident and medical emergency response.

### BACKGROUND

Providing immediate healthcare service to prevent losses caused by accident and to provide immediate assistance during any medical emergency is one of a major challenge faced by multiple countries. At present all accidental and medical emergency facilities are focused towards hospital strengthening but not focused to provide Emergent Health Facility at grass root level during ‘Golden Hour’ period of time to save human life. Moreover, in India most of the hospitals are crowded and when a patient is referred to a hospital, the patient is just unknown about higher health centre so the patient searches to approach the higher health centre by multiple sources. On an average 1.35 million people lose their lives in accidents and more than 15 million people suffer from fatal consequences due to lack of immediate aid. Various systems are available which provide healthcare services.

Conventionally, a system which has been used for providing healthcare services, enables a user to notify manually about an accident or any medical emergency to one or more emergency contacts of the user. However, such systems are unable to provide healthcare services immediately, which causes a loss of lives and economy. Further, another available system, uses a global positioning system for detecting accidents. However, such systems are unable to directly provide any kind of medical emergency aid immediately, which becomes a time-consuming process to aid the victim and unable to prevent fatal consequences. Furthermore, such systems are unable to detect if there is a doctor available at a nearby hospital or if there is a nearby ambulance available for assisting the emergency. Further, such systems require significant human intervention for calling nearby hospitals and nearby available ambulances, which in turn becomes a time-consuming and a life-threatening process.

Hence, there is a need for an improved integrated end to end system and method for accident and medical emergency response in order to address the aforementioned issues.

### BRIEF DESCRIPTION

In accordance with an embodiment of the disclosure, an integrated end to end system for accident and medical emergency response is disclosed. The system includes one or more processors. The system includes a registration subsystem operable by the one or more processors. The registration subsystem is configured to register one or more entities, wherein the one or more entities include one or more affected users and one or more healthcare service providers. The system also includes an emergency determining subsystem operable by the one or more processors. The emergency determining subsystem is configured to determine one or more emergencies associated with one or more affected users registered by the registration subsystem. The system also includes a healthcare service selection subsystem operable by the one or more processors. The healthcare service selection subsystem is configured to enable the one or more affected users to select one or more healthcare services based on the one or more emergencies determined by the emergency determining subsystem. The system also includes a healthcare service provider connecting subsystem operable by the one or more processors. The healthcare service provider connecting subsystem is configured to connect one or more healthcare service providers to the one or more affected users based on the one or more healthcare services selected in the healthcare service selection subsystem. The system also includes a payment receiving subsystem operable by the one or more processors. The payment receiving subsystem is configured to receive a payment from the one or more affected users via a payment card, a cashless healthcare policy or a policy reimbursement for payment upon connecting the one or more healthcare service providers by the healthcare service provider connecting subsystem.

In accordance with another embodiment, a method for providing accident and medical emergency response is disclosed. The method includes registering one or more entities. The method also includes determining one or more emergencies associated with one or more affected users registered by the registration subsystem. The method also includes enabling the one or more affected users to select one or more healthcare services based on the one or more emergencies determined by the emergency determining subsystem. The method also includes connecting the one or more healthcare service providers based on the one or more health care services selected by the one or more affected users in the healthcare service selection subsystem. The method also includes receiving a payment from the one or more affected users via a payment card, the cashless healthcare policy or the policy reimbursement for payment upon connecting the one or more healthcare services by the healthcare service provider subsystem.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a schematic representation of an integrated end to end system for accident and medical emergency response in accordance with an embodiment of the present disclosure;

FIG. 2 is a block diagram of the integrated end to end system for providing accident and medical emergency response of FIG. 1 in accordance with an embodiment of the present disclosure;

FIG. 3 is a block diagram of an embodiment of the integrated end to end system for accident and medical emergency response of FIG. 2 in accordance with an embodiment of the present disclosure;

FIG. 4A, FIG. 4B, FIG. 4C, FIG. 4D, FIG. 4E and FIG. 4F are flow charts representing an embodiment of the integrated end to end system for accident and medical emergency response in accordance with an embodiment of the present disclosure;

FIG. 5 is a block diagram of providing an integrated end to end computer system or a server for accident and medical emergency response in accordance with an embodiment of the present disclosure; and

FIG. 6 is a flow diagram representing steps involved in a method for providing accident and medical emergency response in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to an integrated end to end system and a method for accident and medical emergency response. The system includes one or more processors. The system includes a registration subsystem operable by the one or more processors. The registration subsystem registers one or more entities, wherein the one or more entities include one or more affected users and one or more healthcare service providers. The system also includes an emergency determining subsystem operable by the one or more processors. The emergency determining subsystem determines one or more emergencies associated with one or more affected users registered by the registration subsystem. The system also includes a healthcare service selection subsystem operable by the one or more processors. The healthcare service selection subsystem enables one or more healthcare service providers to select one or more healthcare services based on the one or more emergencies determined by the emergency determining subsystem. The system also includes a healthcare service provider connecting subsystem operable by the one or more processors. The healthcare service provider connecting subsystem connects one or more healthcare service providers based on the one or more health care services selected by the one or more affected users in the healthcare service selection subsystem. The system also includes a payment receiving subsystem operable by the one or more processors. The payment receiving subsystem receives a payment from the one or more affected users via a payment card, the cashless healthcare policy or the policy reimbursement for payment upon connecting the one or more healthcare services by the healthcare service provider subsystem.

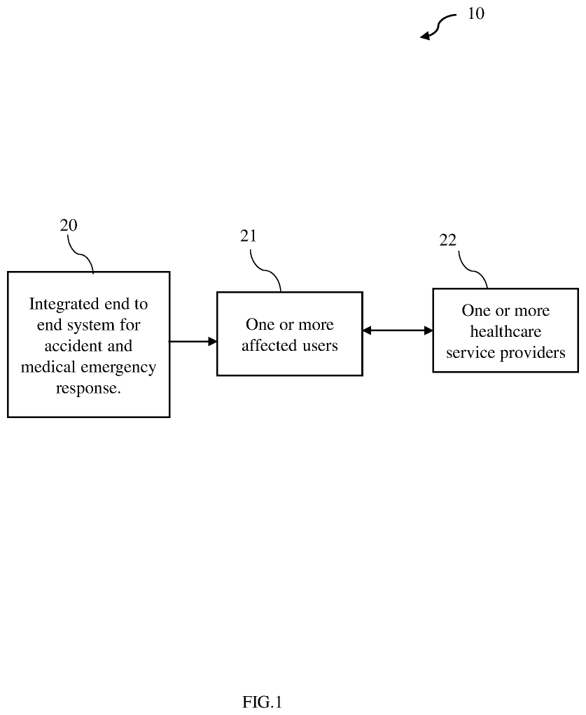

FIG. 1 is a schematic representation of an integrated end to end system (10) for accident and medical emergency response in accordance with an embodiment of the present disclosure. Further, the system (20) enables one or more entities such as one or more affected users (21) and one or more healthcare service providers (22) to register on the system (20) via a mobile device to provide accident and medical response to the one or more affected users (21) by the one or more healthcare service providers (22).

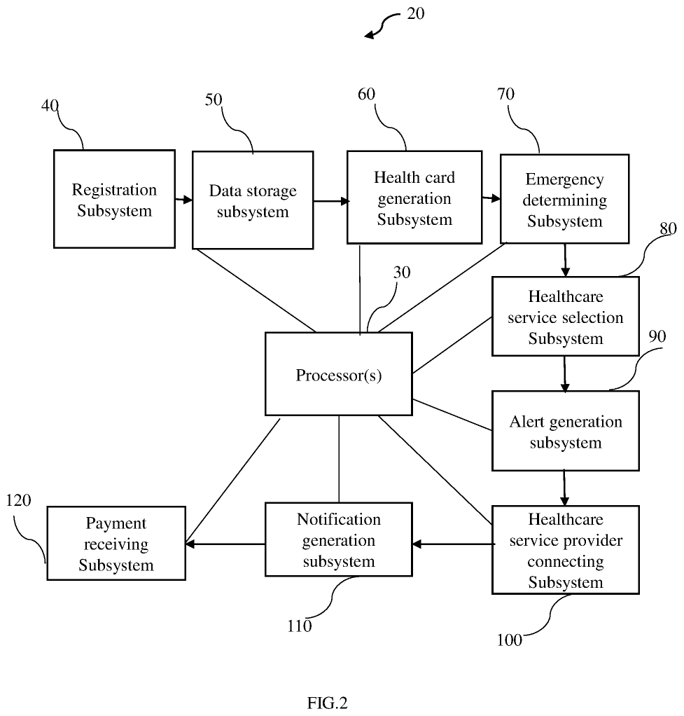

FIG. 2 is a block diagram of the integrated end to end system (20) for accident and medical emergency response of FIG. 1 in accordance with an embodiment of present disclosure. The system (20) includes one or more processors (30). The system (20) also includes a registration subsystem (40) operable by the one or more processors (30). The registration subsystem (40) registers one or more entities via one or more computing devices on a platform. In one embodiment, the one or more entities may include, but not limited to, one or more affected users, one or more bystanders, one or more healthcare service providers and the like. In one embodiment, the one or more computing devices may include, but not limited to, a mobile phone, a tablet, a laptop, and the like. As used herein, the term ‘the one or more affected users’ refers to one or more users who require one or more healthcare services. As used herein, the term ‘the one or more bystanders’ refers to the one or more users standing near to the one or more affected users. In one embodiment, the one or more healthcare service providers may include, but not limited to, an ambulance, police, a fire brigade, a hospital, a health insurance company, a doctor, a nursing staff member, a ward person or a trolley man, a pharmacist and the like.

Further, in some embodiment, the one or more entities may register on the platform by providing one or more details. In one embodiment, the one or more details may include, but not limited to, a name, an address, an age, a blood group, health insurance, an identity proof, a payment card, a health policy details, at least five emergency contact numbers, family tree profile and the like. Further, the system (20) also includes a data storage subsystem (50) operable by the one or more processors (30). The data storage subsystem (50) stores the one or more details associated with the one or more entities registered by the registration subsystem (40). In one embodiment, the data associated with one or more hospitals and one or more hospital staffs are stored in a pre-registered database. In another embodiment, the data associated with the one or more hospitals and the one or more hospital staff is updated regularly after a certain interval of time. In one embodiment, updating data may include, a duty change in hospital staff, a duty change in the police station, a duty change in fire station, a leave or an absent of the one or more hospital staffs and the like based on the duty roaster. In one embodiment, the one or more updates may be done by one or more hospital administrations to assign the one or more emergencies to one or more duty staffs.

In one embodiment, the registration subsystem (40) may enable the one or more affected users to upload a copy of the health policy details, when the one or more affected users have one or more health policies. In one embodiment, the registration subsystem (40) may verify the know your customer (KYC) of a health policy, and also verify and update the approved cashless health facility hospital list to the health profile associated with the one or more affected users for a future cashless treatment facility. In one embodiment, the at least five emergency contact numbers may include, but not limited to, a contact number of family member of the one or more affected users, a contact number of a friend of the one or more affected users and the like.

Further, in one embodiment, the family tree profile may include a profile associated with one or more independent users and a profile associated with one or more dependent users. In such embodiment, the profile associated with the one or more dependent users may include one or more family members of the one or more independent users. In one embodiment, the one or more independent users may share the one or more details with the one or more dependent users. In one embodiment, the independent users may activate the profile associated with the one or more dependent users by giving approval to access the profile associated with the one or more independent users via one or more modes. In some embodiment, the one or more modes may include, but not limited to, an electronic mail, a short message service (SMS), a reference code, a call and the like via an application program interface (API). In another embodiment, the one or more independent users may deactivate the profile of one or more dependent users. In one embodiment, the family tree profile may include a new-born infant profile created by the one or more independent users. In another embodiment, the family tree profile may include a pregnant female user profile from the one or more family members to keep a check on the last menstrual period date (LMP) associated with the pregnant female user.

Further, in one embodiment, the family tree profile may include a special care service associated with the pregnant female user to provide one or more special care updates. In one embodiment, the one or more special care updates may include, a reminder for regular visits, immunization, investigations, one or more prescribed medicines, health record updates and the like. In one embodiment, the family tree profile may also include an update for any change happens in pregnancy and estimated date of delivery (EDD) associated with the pregnant female user. Further, in one embodiment, the new-born infant profile associated with the pregnant female user may be created automatically by the system (20) as after the one or more details updated by the user for one or more changes happened in pregnancy and the estimated date of delivery (EDD), wherein the one or more changes may include, a post-dated delivery or a pre-dated delivery and the like. In another embodiment, the one or more independent users may provide access to the profile associated with the new-born infant to use the payment card for the cashless facility associated with the one or more independent users. In one embodiment, the profile associated with the new-born infant may not be created if the infant dies or a missed abortion and the like. In one embodiment, the information which the one or more affected users wants to share with the one or more hospital service providers may be completely on the discretion of the one or more affected users.

In one embodiment, if the delivery happens before estimated date of delivery (EDD) then instantly the pregnant female user needs to enter details such as a date, a time, a place and the like associated with the delivery and a health status of the pregnant female user. In such embodiment, the profile associated with the infant is self-created by the system (20) and added in the family tree profile.

In some embodiment, hospital or nursing homes may be provided an option specially for pregnant patients that as after entering the delivery date and time in the patients profile with details such as a new born details, weight, physical condition such as alive and the like then the profile associated with the infant will be automatically generated by the system (20) and the like, if any misshapen like death of infant entered to the system (20) then the auto infant profile will not be created and a misshapen is auto recorded in the health record associated with a mother.

Further, in one embodiment, the system (20) may include one or more Aanganwadi workers of one or more government institutes to deal with the delivery of the new-born infant associated with the pregnant female user. In one embodiment, the one or more Aanganwadi workers may feed one or more infant details in the family tree profile after the infant is born. In one embodiment, the one or more infant details may include, but not limited to, a mother name, a father name, date of delivery, place of delivery, time, weight of the infant, a vaccination schedule, mobile number of parents, identity proof of parents and the like. Further, in one embodiment, a profile activation message is sent to the one or more independent users to activate the profile associated with the new-born infant.

Furthermore, in one embodiment, the family tree profile may include a profile associated with a family physician. In one embodiment, the one or more affected users may add one or more details associated with the family physician. In such embodiment, the one or more details associated with the family physician may include, but not limited to, a locality, an area, a city, a speciality, and the like. In one embodiment, the one or more affected users may delete a profile of the family physician associated with a current family physician or add the profile of the family physician associated with new family physician. In one embodiment, the family tree profile may send a notification to the family physician via the one or more modes prior to adding the new family physician profile to the data associated with the family of the one or more affected users.

Further, in some embodiment, the system (20) may include a health card generation subsystem (60) operable by the one or more processors (30). In such embodiment, the health card generation subsystem (60) generates a health card including a health card registration number for the one or more affected users registered by the registration subsystem (40). In one embodiment, the health card registration number may be a unique number corresponding to the one or more affected users. In one embodiment, the health card may be generated by linking with the identity proof of the one or more affected users. In one embodiment, the identity proof may include an Aadhaar card associated with the one or more affected users. In another embodiment, the unique number associated with the health card may never be changed. In one embodiment, the health card may include one or more health details of the one or more affected users.

In such embodiment, the one or more health details may include, but not limited to, a health record, one or more privacy details and the like. In one embodiment, the one or more privacy details may include, but not limited to, name of one or more users who have rights to access the health record, lab reports and the like. In one embodiment, the one or more users, may include, but not limited to, the one or more family members, the family physician, the hospital, and the like. In one embodiment, the one or more affected users may select the name of the one or more users to access the health record of the one or more affected users. In one embodiment, the one or more health details may be corrected and never be deleted and further record one or more health card details in the system (20). In such embodiment, correction of the one or more health details may be recorded with a date, a time, by whom (a name, a designation and a login identity) associated with the correction of the one or more health details for security and privacy and to identify any misuse.

In one embodiment, the health card generation subsystem (60) may maintain a record of one or more health events, wherein the one or more health events may include, a OPD consultations, a hospital admissions, surgeries, medications, a diagnosis, reports and the like associated with the health card of the one or more users. In one embodiment, the one or more updates may include, but not limited to, a login with name, a time of login, a date and the like. In one specific embodiment, the one or more health care service providers may access the health record after one or more validations or verifications. In such embodiment, the one or more validations and verification may include, a know your customer (KYC) of practitioners, the hospital, a clinic legal registration, a qualification registration of a healthcare worker from an authority and the like. In one embodiment, the one or more affected users may restrict and permit the one or more users to access the health profile associated with the one or more affected users. In one embodiment, one or more access to the health record may be under the strict vigil eye of the profile associated with the one or more users. In one embodiment, the one or more access may be recorded by the system (20) with a date, a time, an identity associated with the users accessing the profile and the like. In one specific embodiment, the one or more users' self-access may also be recorded for safety and security of the one or more details associated with the one or more users.

Further, the system (20) includes an emergency determining subsystem (70) operable by the one or more processors (30). The emergency determining subsystem (70) determines one or more emergencies associated with the one or more affected users registered by the registration subsystem (40). In one embodiment, the one or more emergencies may include, but not limited to, a road traffic accident, a referral, and the like. In one embodiment, the emergency determining subsystem (70) may enable the one or more entities to upload a picture of the one or more affected users to determine at least one emergency from the one or more emergencies. In one embodiment, the emergency determining subsystem (70) may include an emergency panic subsystem operable by the one or more processors (30). The emergency panic subsystem enables the one or more affected users to get the healthcare service without getting registered by the registration subsystem (40) in case of an emergency. In one embodiment, the emergency panic subsystem may confirm the one or more affected users via a one-time password (OTP) for verification of the one or more affected users. In one embodiment, the emergency determining subsystem may include one or more end to end facilities, wherein the one or more end to end facilities refer to intimate a current status from the start to end of the treatment up to the discharge or a final outcome of the treatment associated with the one or more affected users to update continuously.

Further, in one embodiment, the referral from the one or more emergencies may include a referral system from rural areas (a primary health centre) to a secondary health centre and from the secondary health centre to a tertiary health centre for the treatment of the one or more affected users. In one embodiment, one or more referred affected users from one health centre to a higher health centre are provided with an ambulance service as well as a prior emergency appointment will be fixed with the higher health centre. In one embodiment, a current health condition and reason for a refer and copy of a referral card associated with the one or more affected users may be shared with the higher health centre so that as soon as the one or more affected users reaches to a referred hospital for getting treatment with provisional diagnosis shared earlier via the health record. In one embodiment, the higher health centre may be notified about the estimated arrival time with a live GPS location of the one or more affected users with current condition, so that the referred hospital can arrange the facilities accordingly.

Further, the system (20) also includes a healthcare service selection subsystem (80) operable by the one or more processors (30). The healthcare service selection subsystem (80) enables the one or more affected users to select one or more healthcare services based on the one or more emergencies determined by the emergency determining subsystem (70). In one embodiment, the one or more healthcare services may include, but not limited to, an ambulance service, a hospital service, a health worker service, a police service, a fire service and the like. In one embodiment, the system may include an alert generation subsystem (90) operable by the one or more processors (30). In such embodiment, the alert generation subsystem (90) generates an alert signal for the one or more healthcare service providers via the one or more modes upon selection of the one or more health care services by the healthcare service selection subsystem (80). In one embodiment, the one or more modes may include, but not limited to, an SMS, an electronic mail and the like.

Furthermore, in one exemplary embodiment, the one or more affected users or one or more bystanders may select at least one of a police service, the fire service or the ambulance service from the one or more healthcare services corresponding to the one or more emergencies determined by the emergency determining subsystem (70). In such embodiment, the one or more affected users or the one or more bystanders may capture one or more site details of the one or more affected users for sending the one or more site details to the at least one of the ambulance service, the police service or the fire service. In one embodiment, the one or more site details may include, but not limited to, a mobile number of calls received, a location of the accident, and the like. Further, in one embodiment, the at least one of the ambulance service, the police service or the fire service navigates a location of the one or more affected users using a global positioning system (GPS) upon receiving the one or more site details from the one or more affected users or the one or more bystanders.

Further, the system (20) also includes a healthcare service provider connecting subsystem (100) operable by the one or more processors (30). The healthcare service provider connecting subsystem (100) connects the one or more healthcare service providers to the one or more affected users based on the one or more healthcare services selected in the healthcare service selection subsystem (80). In one exemplary embodiment, the healthcare service provider connecting subsystem (100) may connect the at least one of the ambulance service, the police service or the fire service to the one or more affected users upon navigating a location of the one or more affected users.

In one exemplary embodiment, the ambulance service, the police service or the fire service fills data associated with the one or more affected users and upload one or more pictures of the one or more affected users to connect to one or more near-by hospital services from the one or more healthcare services. In one embodiment, the ambulance service may search a nearby ambulance using a global positioning system (GPS) via an application program interface (API) to locate the nearby government ambulance and if the government ambulance is not available or if the government ambulance is far away then the ambulance service uses the GPS navigation of a nearby private ambulance. In one scenario, the private ambulance may navigate the location of the one or more affected users via the GPS associated with the ambulance. In another embodiment, the location of the one or more affected users may be navigated by using the mobile GPS associated with the duty driver of the private ambulance. In one embodiment, the system (20) updates for the driver duty whenever the driver is changed in the private ambulances.

Further, in some embodiment, the system (20) may include a notification generation subsystem (110) operable by the one or more processors (30). The notification generation subsystem (110) generates one or more notification signals for the one or more healthcare service providers upon connecting the one or more healthcare service providers by the healthcare service provider connecting subsystem (100). In one embodiment, the notification generation subsystem (110) may generate notification for the at least five emergency contact numbers, the hospital, the ward service, the trolley man, the nurse, the pharmacist, the duty doctor, the observation ward, and the like. In one embodiment, the notification generation subsystem (110) notifies the ward person from the one or more healthcare service providers to hand over the one or more affected users to the hospital and to drop the one or more affected users after the one or more affected users are discharged as per as one or more received instructions. In one embodiment, the one or more instructions may include, an instruction by a doctor, a transfer destination such as discharge from an emergency or shift to the observation ward, Intensive Care Unit (ICU) or General ward and the like. In one embodiment, one or more transfer updates may be notified to the user as well as to the next defined destination ward for further compliance and the treatment. In one embodiment, the system (20) continuously track the patient treatment status until the discharge or final treatment outcome. In one embodiment, the final treatment outcome along with the invoice and payment receipt details may be notified to the user via Short message service (SMS) or a mobile app notification.

In some embodiment, the notification generation subsystem (110) may notify the nurse to enter one or more treatments given to the one or more affected users for updating the health record of the one or more affected users via a voice note dictation or a text dictation. Further, in one embodiment, the notification generation subsystem (110) may notify the ward service about one or more updates associated with the one or more affected users. In one embodiment, the one or more updates may include, but not limited to, a transfer, a next duty, a duty completion, and the like. In one embodiment, the notification generation subsystem (110) notifies the duty doctor to handle the one or more affected users and to enter one or more treatment details. In one embodiment, the one or more treatment details may include a treatment slip image, recovered updates, discharge updates, operation lab updates and the like. In one embodiment, the one or more treatment details will be received by the one or more affected users and the nursing staff. In one embodiment, the notification generation subsystem (110) may also notify the duty doctor, the nursing attendant, a hospital administration and the one or more affected users if the one or more affected users is shifted to any next step such as an observation ward, Intensive Care Unit (ICU) or General ward for the further compliance and treatment and the like. In one embodiment, the system (20) may continuously track the patient treatment status until the discharge or the final treatment outcome.

In one specific embodiment, the one or more affected users may select a routine healthcare service from the one or more healthcare services. In one embodiment, the routine healthcare service may include an out-care patient department (OPD) or an in-care patient department (IPD). In some embodiment, the one or more affected users may select one or more facilities from the one or more healthcare services upon selecting the OPD or the IPD from the one or more healthcare services.

Further, in one embodiment, the one or more facilities may include, but not limited to, a doctor, a specialist, an appointment time, a hospital, and the like. In one embodiment, the routine healthcare service may generate a personalised daily report for the one or more affected users. In one embodiment, the one or more affected users may get a special facility when the age of the one or more affected users exceed a predefined age. In such embodiment, the one or more special facilities may include, a separate que, a quick check up and the like.

Further, in one embodiment, one or more senior affected users may be given a special priority based on TRIAGE system. As used herein, the term ‘TRIAGE system’ refers to a special and urgent appointment system special for the one or more senior affected users during the one or more emergencies. In one embodiment, the one or more senior affected users may include a separate queue to facilitate a special care for the one or more senior affected users. In one embodiment, the TRIAGE system may include one or more categories. In one embodiment, the one or more categories may include, an expectant category in black colour, an immediate category in red colour, a delayed category in yellow colour, a minor category in green colour and the like.

Furthermore, the system (20) includes a payment receiving subsystem (120) operable by the one or more processors (30). The payment receiving subsystem (120) receives a payment from the one or more affected users via a payment card upon connecting the one or more healthcare service providers by the healthcare service provider subsystem (100). In one embodiment, the payment card may be shared with one or more family members of the one or more affected users upon receiving approval. In one embodiment, the payment card may include, but not limited to a debit card, a credit card and the like. In one embodiment, the payment receiving subsystem (120) may connect to one or more healthcare policy provider for payment if the one or more affected users includes a health care policy and during the user registration know your customer (KYC) of health policy the system (20) may verify and update the approved cashless health facility hospital list to the user's health profile for future cashless treatment facility. In one embodiment, the one or more affected users may receive a discharge summary along with the payment. In another embodiment, if the one or more affected users has a health insurance then an invoice may be generated and send to an insurance company associated with the one or more affected users to claim the health insurance.

In one specific embodiment, the system (20) may include an analysing subsystem operable by the one or more processors (30). The analysing subsystem is configured to analyse the working of one or more government health systems. In one embodiment, the one or more government health systems may include, a government health institution, a government health centre and the like. In one embodiment, the analysing subsystem analyses negligence in the working of the one or more government health systems.

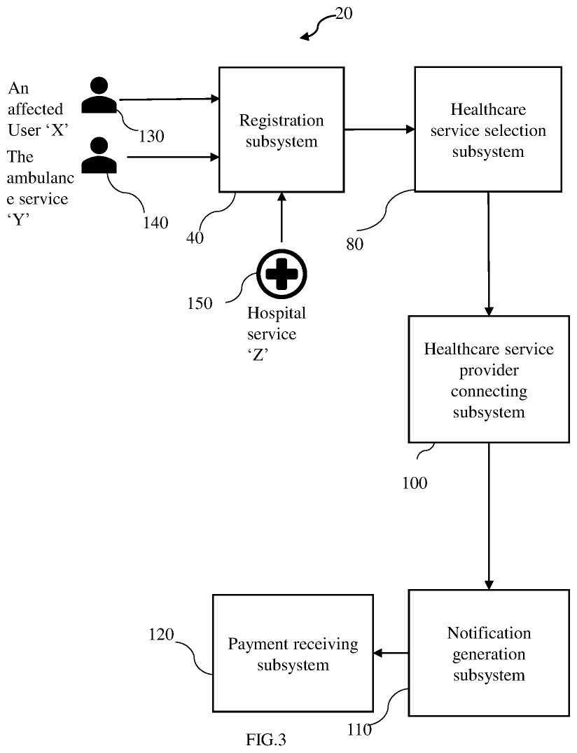

FIG. 3 is a block diagram of an embodiment of the system (20) for accident and medical emergency response of FIG. 2 in accordance with an embodiment of the present disclosure. An affected user ‘X’ (130), an ambulance service ‘Y’ (140) and a hospital service ‘Z’ (150) registers on a platform, by a registration subsystem (40), by providing multiple details including an age, a name, a health record, and one or more details associated with the affected user ‘X’ (130) using a mobile phone. Upon registration on the platform, the affected user ‘X’ (130) selects the ambulance service ‘Y’, by a healthcare service selection subsystem (80). Further, in one case, the affected user ‘X’ (130) gets connected with the ambulance service ‘Y’ (140), the hospital service ‘Z’ (150) and the ambulance service from the one or more healthcare services selected by the affected user ‘X’, by a healthcare service provider connecting subsystem (100).

Furthermore, a notification signal for the ambulance service ‘Y’ (140) gets generated to further connect with the hospital service ‘Z’ (150) associated with the affected user ‘X’ (130), by a notification generation subsystem (110). Furthermore, the affected user ‘X’ (130) receives a bill amount of the ambulance service ‘Y’ (130) and the hospital service ‘Z’ (150) from the one or more healthcare services, by a payment receiving subsystem (120).

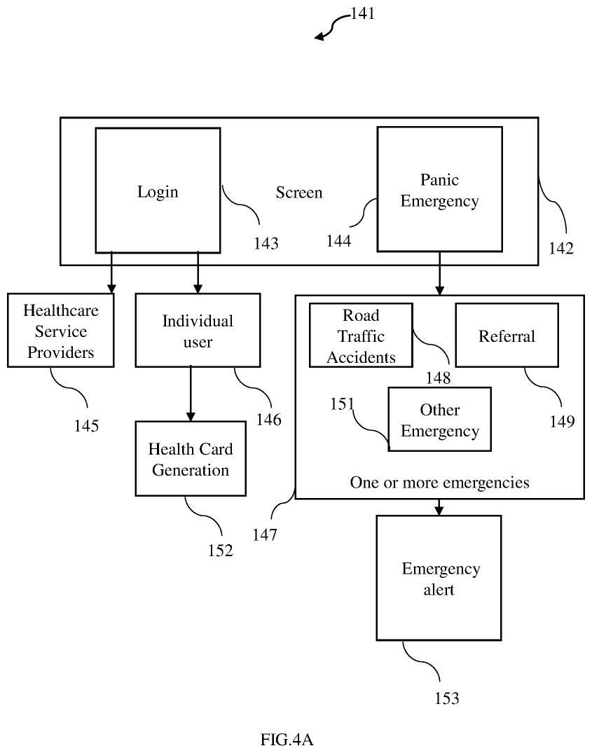

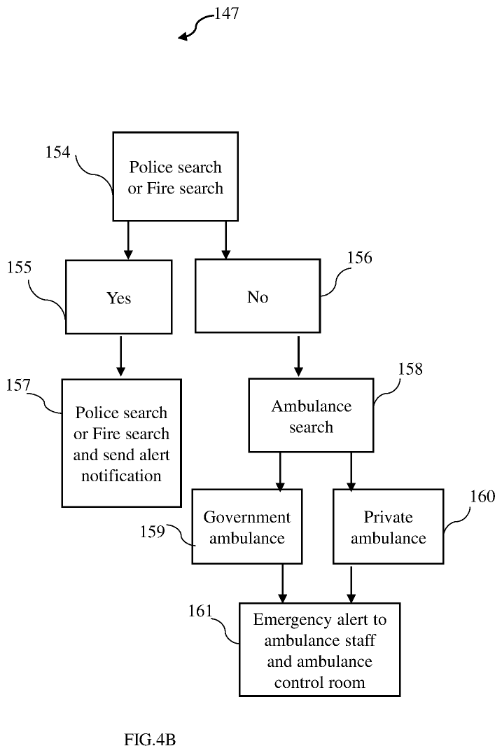

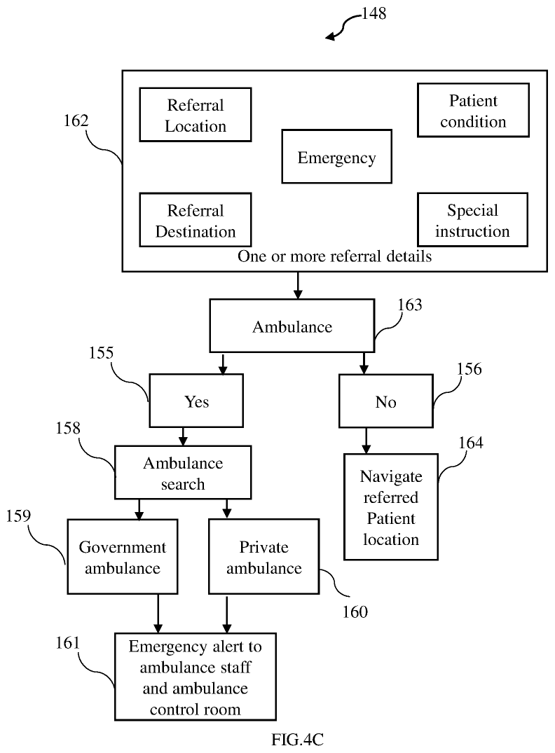

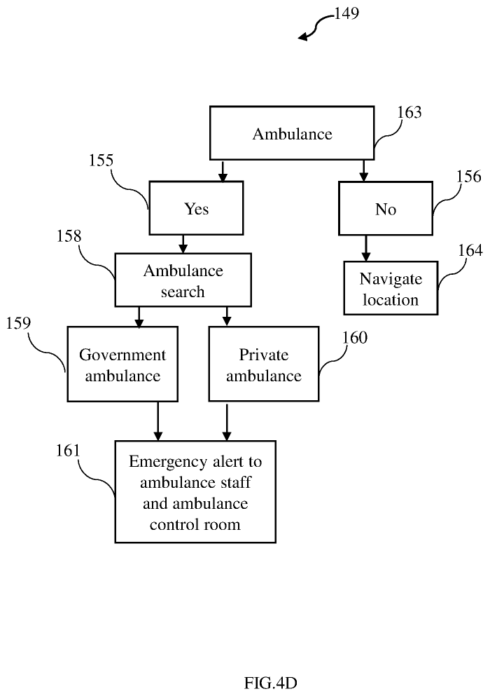

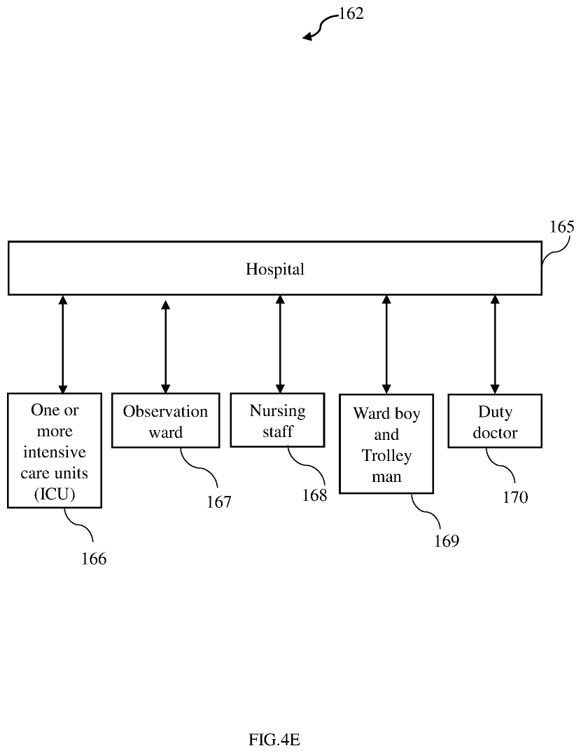

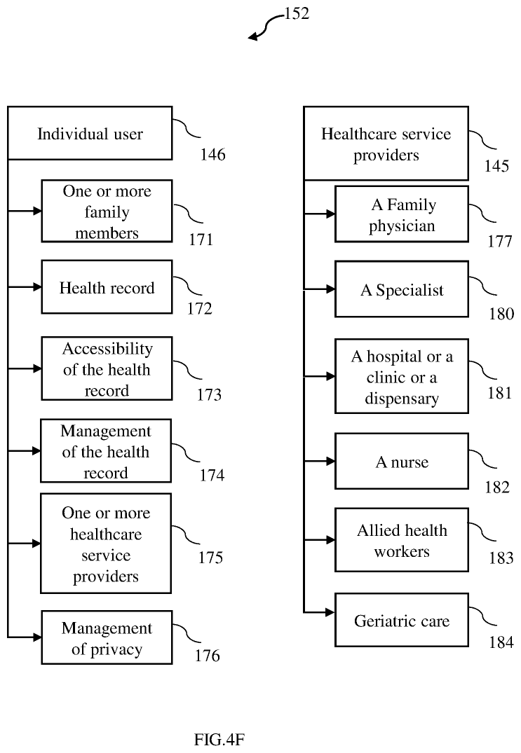

FIG. 4A, FIG. 4B, FIG. 4C, FIG. 4D, FIG. 4E, and FIG. 4F are flow charts representing an embodiment of the system (20) for accident and medical emergency response of FIG. 2 in accordance with an embodiment of the present disclosure. In one scenario, an individual user (146) and one or more healthcare service providers (145) register on a platform via a screen (142) by selecting a login option from the platform in step 142, wherein the one or more healthcare service providers (145) may include, but not limited to, a hospital, a doctor, a nursing staff, a ward boy or a trolley man, an ambulance service, or healthcare service administrator, a pharmacist, a lab technician, a police service, a fire service and the like.

Upon registering, the individual user (146) provides one or more details, wherein the one or more details may include, but not limited to, a name, an address associated with the individual user (146), a mobile number, an age, a blood group, a health insurance, an identity proof, a payment card, a health policy detail, a list of five emergency contact numbers, a vehicle detail associated with the individual user (146). Further, the system (20) verifies an identity of the individual user (146) via the identity proof. Upon providing the one or more details the individual user (146) fills the health policy details and a copy of the health policy for verification from the health policy provider (145). Further, the system (20) verifies and update the approved cashless health facility hospital list to the health profile associated with the user for future cashless treatment facility. Furthermore, the system (20) verifies the payment card associated with the individual user (146) through a one-time password (OTP) via the mobile number from the one or more details provided by the individual user (146).

Further, the list of five emergency contact numbers associated with the individual user (146) receives a consent notification to permit the individual user (146) to register an emergency contact number in the list of five emergency contact numbers. Upon permitting the individual user (146), the list of five emergency contact numbers receive one or more alerts via one or more modes, wherein the one or more alerts may include, but not limited to, a location of the individual user (146), an accident pic associated with the individual user (146) and the like. Further, the one or more modes may include a call, a short message service (SMS) and the like.

Upon receiving the vehicle details associated with the individual user (146), the system (20) gives one or more reminders to the individual user (146) associated with the vehicle, wherein the one or more reminders may include, a renewal of insurance, a pollution control check-up and the like. Further, upon registering and verifying the one or more details of the individual user (146), a health card is generated with a unique health card number and a unique health card bar code associated with the individual user (146) in step 152. Furthermore, the individual user (146) also creates family tree profile for one or more family members associated with the individual user (146) to avail a cashless payment facility via the payment card of the individual user (146). Upon filling one or more names and a relation of the one or more family members with the individual user (146) the one or more family members receive a notification with a link to register on the platform by entering a reference code. Upon registering by the one or more family members, the one or more details of the one or more family members will be fetched from the one or more details associated with the individual user (146). After registering and fetching the one or more details of the one or more family members, an activation notification of one or more profiles associated with the one or more family members is received by the individual user (146).

Further, upon creating the family tree profile, the individual user (146) creates a family physician profile, wherein the individual user (146) registers a family physician by providing one or more details associated with the family physician, wherein the one or more details may include, a location of the family physician, a name, an address, a specialty and the like. Upon registering the family physician profile, the family physician will approve a consent message for an approval to be listed as the family physician of the individual user (146). Further, the individual user (146) may delete an existing family physician profile or add a new family physician profile. Further, upon registering the family physician profile, the individual user (146) and the one or more family members associated with the individual user (146) communicate with the associated family physician for one or more medical needs.

Further, the individual user (146) creates a new family member profile for an infant to generate a health card (152), wherein the health card (152) will start from the birth of the infant to one or more updates associated with one or more health visits in one or more hospitals, wherein the one or more updates may include, but not limited to, one or more vaccines updates, a discharge update and the like.

Further, in another scenario, the system (20) also provides the individual user (146) a single time emergency access without registering by selecting a panic emergency option from the screen in step 144, wherein the individual user (146) enters a mobile number Upon entering the mobile number, the individual user (146) receives an onetime password for verification of the individual user (146). Upon verification of the individual user (146), the individual user (146) selects at least one emergency from one or more emergencies (147), wherein the one or more emergencies (147) may include, but not limited to, a road traffic accident (148), a referral (149), other emergencies (151) and the like in step 147. Upon selection of the at least one emergency from the one or more emergencies (147), the individual user (146) captures and uploads an image associated with the at least one emergency from the one or more emergencies (147). Furthermore, upon capturing and uploading the image, the list of five emergency contacts receives an emergency alert associated with the at least one emergency from the one or more emergencies (147) of the individual user (146) in step 153.

Further, in a first case, the individual user (146) selects a road traffic accident (148) from one or more emergencies in step 147. Upon selecting the road traffic accident (148), the individual user (146) selects at least one option from one or more options, wherein the one or more options may include at least one of a police search, a fire search, a combination thereof in step 154. Further, the individual user (146) selects at least one of a police search, a fire search or a combination thereof from the one or more options then the system (20) starts searching for at least one of a nearby police station, a nearby fire station or a combination thereof using a google map in step 157. Furthermore, if the individual user (146) selects none from the one or more options then the system (20) directs the individual user (146) to an ambulance search page.

In a second case, if the individual user (146) selects “yes” option from the ambulance search page then the system (20) will search one or more nearby ambulances according to the availability of the one or more nearby ambulances in step 158, wherein the one or more ambulances may include, a government ambulance (159), a private ambulance (160) and the like. Upon searching for a nearby ambulance, the system (20) sends an emergency alert notification associated with the individual user (146) to one or more ambulance staff in step 161, wherein the one or more ambulance staff may include, a driver, a nurse and the like.

Upon sending the emergency alert notification, the nearby ambulance reaches the location of the individual user (146) and feed one or more details associated with the individual user (146) to start searching a nearby hospital, wherein the one or more details may include, an image of the individual user (146), a name and the like. In a third case, if the individual user (146) selects no option from the ambulance search page then the system (20) starts to navigate a mobile location associated with the individual user (146) and establish a communication between the hospital (181) and the one or more users or the one or more ambulance staff to communicate with an assigned doctor from the hospital during a transit of the individual user (146).

Moreover, in first case, an individual user (146) selects a referral option from one or more emergencies (147) in step 149. Upon selecting, the individual user (146) feeds one or more referral details in step 162, wherein the one or more referral details may include, a referral location, a patient condition, a referral destination, an emergency, a special instruction and the like. Upon feeding, the system (20) takes the individual user (146) to an ambulance option page in step 163. If the individual user (146) selects yes (155) option from the ambulance search page then the system (20) will search one or more nearby ambulances according to the availability of the one or more nearby ambulances in step 158, wherein the one or more ambulances may include, a government ambulance (159), a private ambulance (160) and the like. Upon searching a nearby ambulance, the system (20) sends an emergency alert notification associated with the individual user (146) to one or more users or the one or more ambulance staff in step 161, wherein the one or more ambulance staff may include, a driver, a nurse and the like.

Upon sending the emergency alert notification, the nearby ambulance reaches the location of the individual user (146) and feed one or more details associated with the individual user (146) to start searching a nearby hospital, wherein the one or more details may include, an image of the individual user (146), a name and the like. In third case, if the individual user (146) selects no (156) option from the ambulance search (158) page then the system (20) starts to navigate a mobile location associated with the individual user (146) in step 164 and establish a communication between the hospital (181) and the one or more affected users to communicate with an assigned doctor from the hospital (181) during a transit of the individual user (146).

Moreover, in first case, an individual user (146) selects other emergency options from one or more emergencies (147) in step 150. After selecting, the system (20) takes the individual user (146) to an ambulance option page in step 163. In second case, if the individual user (146) selects yes (155) option from the ambulance search page then the system (20) will search one or more nearby ambulances according to the availability of the one or more nearby ambulances in step 158, wherein the one or more ambulances may include, a government ambulance (159), a private ambulance (160) and the like. Upon searching a nearby ambulance, the system (20) sends an emergency alert notification associated with the individual user (146) to one or more ambulance staff in step 161, wherein the one or more ambulance staff may include, a driver, a nurse and the like.

Upon sending the emergency alert notification (161), the nearby ambulance reaches the location of the individual user (146) and feed one or more details associated with the individual user (146) to start searching a nearby hospital, wherein the one or more details may include, an image of the individual user (146), a name and the like. In the third case, if the individual user (146) selects no (156) option from the ambulance search (158) page then the system (20) starts to navigate a mobile location associated with the individual user (146) in step 164 and establish a communication between the hospital (181) and the one or more users or one or more patients attendants from the one or more ambulance staffs to communicate with an assigned doctor from the hospital during a transit of the individual user (146).

Further, upon selection of a hospital (165), the system (20) navigates the individual user (146) to the hospital (181) and simultaneously sends an alert to one or more hospital staff so that the one or more hospital staffs receives an expected individual user (146) on time in step 162, wherein the one or more hospital staffs may include, a duty doctor (170), a nursing staff (168), a ward boy (169), one or more intensive care unit (ICU) (166), an observation ward (167) and the like. Upon reaching the hospital (165), an alert associated with the individual user (146) about reaching the hospital is generated for the hospital (165). Upon receiving, the system (20) will show the individual user (146) active and under treatment by the one or more hospital staff.

Furthermore, upon registering, a health card (152) is generated with a unique health bar code number, a unique health identification number and an image associated with the individual user (146) in step 152, wherein the unique health bar code and the unique health identification number carries forward, accepted and identified in all the hospitals, Ambulance providers and other registered health care service providers. The individual user (146) may go for one or more treatments in one or more hospitals, the individual user (146) needs to give only health card (152) to the hospital staff and the hospital staff will scan the unique health bar code or enter the unique health Identification number then one or more details of the individual user (146) are automatically fetched.

In such embodiment, the individual user (146) may not carry one or more documents like the one or more details associated with the individual user (146) will automatically be provided by the system. In another embodiment, one or more health visits, diagnosis, investigations and treatment records associated with the individual user (146) are automatically recorded to health profile associated with the individual user (146) each and every time, day, time, duration. In one embodiment, the one or more documents may include, an identity proof, one or more previous hospital records and the like. Further, one or more hospital details may be entered by the duty doctor or a duty staff. In one embodiment, the one or more hospital details may include, further instructions of treatment, a shift to a ward or ICU, a discharge and the like. Upon entering the one or more hospital details then the individual user (146) will be shown active in next destination and all further treatment record will be maintained by concerned staff of that ward until discharge and the system (20) will only show the treatment completed when a summary associated with the individual user (146) or an outcome associated with the treatment is updated in the system (20).

Further, the individual user (146) selects one or more family (171) members associated with the individual user (146) to access the health card (152). In one scenario, the health record (172) of the individual user (146) is also stored in the health card (152) and the individual user (146) manages the health record (172), one or more healthcare service providers (145) and privacy associated with the individual user (146). In another scenario, the one or more healthcare service providers (145) can access the health record (172) associated with the individual user (146), wherein the one or more health care service providers (145) may include, a family physician (177), a specialist (180), a hospital (181), a nurse (182), allied health workers (183), a geriatric care (184) and the like.

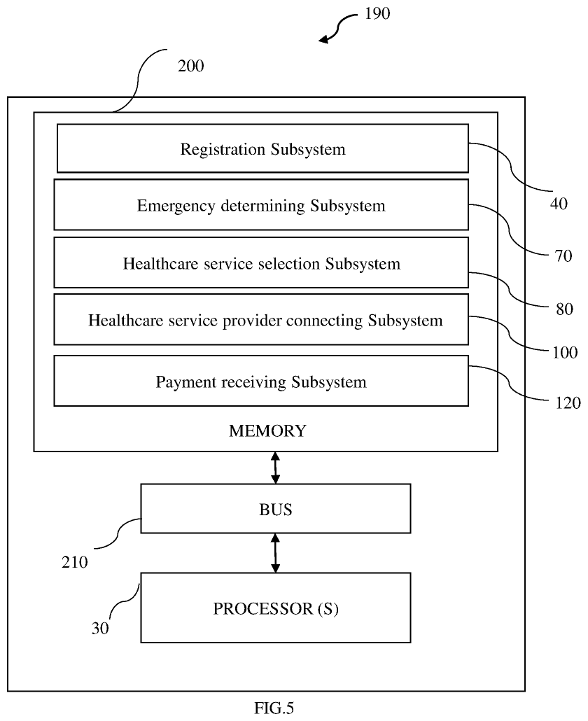

FIG. 5 is a block diagram of an integrated end to end computer system or server (190) in accordance with an embodiment of the present disclosure. The computer system (190) includes processor(s) (30), and memory (200) coupled to the processor(s) (30) via a bus (210). The memory (200) is stored locally on a user device.

The processor(s) (30), as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory (200) includes a plurality of units stored in the form of executable program which instructs the processor (30) to perform the configuration of the system illustrated in FIG. 1. The memory (200) has following subsystems: a registration subsystem (40), an emergency determining subsystem (70), a healthcare service selection subsystem (80), a healthcare service provider connecting subsystem (100) and a payment receiving subsystem (120) of FIG. 2.

Computer memory (200) elements may include any suitable memory device(s) for storing data and executable program, such as read-only memory, random access memory, erasable programmable read-only memory, electrically erasable programmable read-only memory, hard drive, removable media drive for handling memory cards and the like. Embodiments of the present subject matter may be implemented in conjunction with program subsystems, including functions, procedures, data structures, and application programs, for performing tasks, or defining abstract data types or low-level hardware contexts. The executable program stored on any of the above-mentioned storage media may be executable by the processor(s) (30).

The registration subsystem (40) instructs the processor(s) (30) to register one or more entities. The emergency determining subsystem (70) instructs the processor(s) (30) to determine one or more emergencies associated with the one or more affected users registered by the registration subsystem (40). The healthcare service selection subsystem (80) instructs the processor(s) (30) to enable the one or more affected users to select one or more healthcare services based on the one or more emergencies determined by the emergency determining subsystem (70).

The healthcare service provider connecting subsystem (100) instructs the processor(s) (30) to connect the one or more healthcare service providers to the one or more affected users based on the one or more healthcare services selected in the healthcare service selection subsystem (80). The payment receiving subsystem (120) instructs the processor(s) (30) to receive a payment from the one or more affected users via a payment card upon connecting the one or more healthcare service providers by the healthcare service provider connecting subsystem (100).

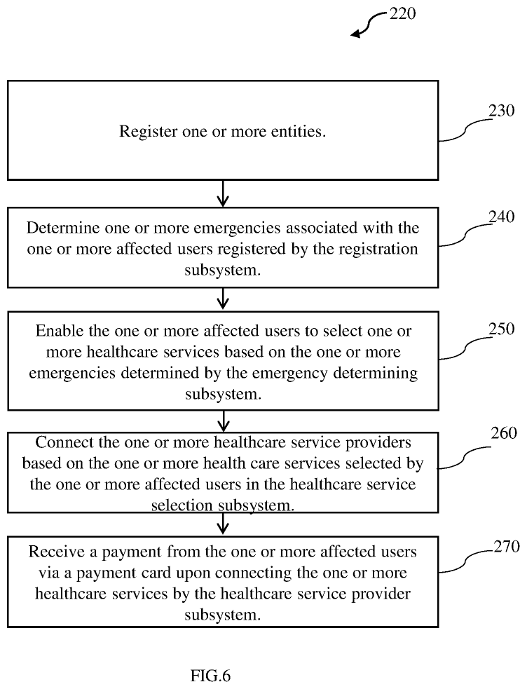

FIG. 6 is the flow diagram representing steps involved in a method (220) for accident and emergency response in accordance with an embodiment of the present disclosure. The method (220) includes registering, by a registration subsystem, one or more entities in step 230. In one embodiment, registering the one or more entities may include registering the one or more entities via one or more computing devices on a platform. In one embodiment, registering the one or more entities may include registering one or more affected users, one or more bystanders, one or more healthcare service providers and the like. Further, in such embodiment, registering the one or more healthcare service providers include registering an ambulance, police, a fire brigade, a hospital, a health insurance company, a doctor, a nursing staff member, a ward person or a trolley man, a pharmacist and the like.

In one embodiment, registering the one or more entities via the one or more computing devices may include registering the one or more users via a mobile phone, a tablet, a laptop, and the like. In one embodiment, registering the one or more entities on the platform may include registering the one or more entities on the platform by providing one or more details. In one embodiment, providing the one or more details may include providing a name, an address, an age, a blood group, health insurance, an identity proof, a payment card, a health policy details, at least five emergency contact numbers, data associated with a family and the like.

Further, the method (220) may include storing, by a data storage subsystem, the one or more details associated with the one or more entities registered by the registration subsystem In one embodiment, the method (220) may include enabling, by the registration subsystem, the one or more affected users to upload a copy of the health policy details if the one or more affected users have one or more health policies. In one embodiment, the method (220) storing, data associated with one or more hospitals and one or more hospital staff, in a pre-registered database. In one embodiment, the method (220) may include updating, the data associated with the one or more hospitals and the one or more hospital staffs are regular with one or more updates. In one embodiment, updating the one or more updates may include updating a duty change in hospital staff, a duty change in the police station, a duty change in fire station, a leave or an absent of the one or more hospital staffs and the like. In one embodiment, updating the one or more updates may be done by one or more hospital administrations to assign the one or more emergencies to one or more duty staffs.

In one embodiment, providing the family tree profile may include providing a profile associated with one or more independent users and a profile associated with one or more dependent users. In one embodiment, the profile associated with the one or more dependent users may include the profile associated with one or more family members of the profile associated with the one or more independent users. In one embodiment, the method (220) may include sharing, by the one or more independent users, the one or more details with the one or more dependent users. In one embodiment, the method (220) may include activating, by the one or more independent users, the profile associated with the one or more dependent users by giving approval to access the profile associated with the one or more independent via one or more modes. In one embodiment, activating the profile associated with one or more dependent users via the one or more modes may include activating the profile associated with one or more dependent users via an electronic mail, a short message service (SMS), a reference code and the like. In one embodiment, the method (220) may include creating, by the one or more independent users, a profile associated with a new infant. In one embodiment, the method (220) may include keeping, by the family tree profile, a check on the last menstrual period date (LMP) associated with the pregnant female user.

Further, in one embodiment, the method (220) may include associating, by the family tree profile, a special care service with the pregnant female user to provide one or more special care updates. In one embodiment, associating the special care may include associating a reminder for regular visits, immunization, investigations, one or more prescribed medicines, health record updates and the like. In one embodiment, updating, by the family tree profile, any change happens in pregnancy and estimated date of delivery (EDD) associated with the pregnant female user. In on embodiment, the method (220) may include creating, by the system (20), an infant profile associated with the pregnant female user automatically. In another embodiment, the method (220) may include providing, by the one or more independent users, access to the profile associated with the infant to use the payment card for the cashless facility associated with the one or more independent users. In one embodiment, the method (220) may include not creating, by the system (20), the profile associated with the infant if the infant dies.

In one embodiment, the method (220) may include entering details such as a date, a time, a place and the like associated with the delivery and a health status of the pregnant female user if the delivery happens before EDD. In such embodiment, the method (220) may include creating the profile associated with the infant by the system (20) and added in the family tree profile. In one embodiment, the method (220) may include providing hospital or nursing homes as an option specially for pregnant patients that as after entering the delivery date and time in the patients profile with details such as a new born details, weight, physical condition such as alive, then an auto infant profile is generated by the system (20) and the like, if any misshapen like death of infant entered to the system (20) then the auto infant profile will not be created and misshapen is auto recorded in the mother's health card details as the health record.

In one embodiment, the method (220) may include providing, one or more Aanganwadi workers of government institutes to deal with the delivery of the infant associated with the pregnant female user. In one embodiment, the method (220) may include feeding, by the one or more Aanganwadi workers one or more infant details in the family tree profile after the infant is born. In one embodiment, feeding the one or more infant details may include feeding, mother name, father name, date of delivery, place of delivery, time, the weight of the infant, a vaccination schedule, mobile number of parents, identity proof of parents and the like. Further, in one embodiment, the method (220) may include sending, by the family tree profile, a profile activation message to the one or more independent users to activate the profile associated with the infant.

In one embodiment, the method (220) may include adding, by the one or more affected users, one or more details associated with a family physician. In one embodiment, adding the one or more details associated with the family physician may include adding, a locality, an area, a city, a specialty, and the like. In one embodiment, the method (220) may include deleting, by the one or more affected users, a profile associated with a current family physician or adding a profile associated with a new family physician. In one embodiment, the method (220) may include sending, by the family tree profile, a notification to the family physician via the one or more modes prior to adding the profile associated with the new family physician profile to the data associated with the family of the one or more affected users.

Further, the method (220) may include generating, by a health card generation subsystem, a health card comprising a health card registration number for the one or more affected users registered by the registration subsystem. In one embodiment, generating the health card including the health card registration number may include generating the health card including a unique number corresponding to the one or more affected users. In one embodiment, generating the health card may include generating the health card by linking with the identity proof, of the one or more affected users. In one embodiment, generating by the identity proof may include generating by Aadhaar card associated with the one or more affected users.

In one embodiment, the method may include providing, by the health card, one or more health details of the one or more affected users. In such embodiment, providing the one or more health details may include a health record, one or more privacy details and the like. In one embodiment, providing the one or more privacy details may include providing the health record, names of one or more users who have rights to access the health record associated with one or more users, lab reports, and the like. In one embodiment, the method (220) may include selecting, by the one or more affected users, the name of the one or more users to access the health record of the one or more affected users. In one embodiment, the method (220) may include correcting and never deleting the one or more health card details and further recording the one or more health card details in the system (20). In such embodiment, correction of the one or more health card details may be recorded with a date, a time associated with the correction of the one or more health details for security and privacy and to identify any misuse.

In one embodiment, the method (220) may include maintaining, a record of one or more updates associated with the health card of the one or more users. In one embodiment, the one or more updates may include, but not limited to, a login with name, a time of login, a date and the like. In one specific embodiment, the method (220) may include accessing the health record after one or more validations. In such embodiment, accessing the one or more validations may include, accessing a know your customer (KYC) of practitioners, the hospital, a clinic legal registration, a qualification registration from an authority and the like. In one embodiment, the method (220) may include restricting and permitting, by the one or more affected users, the one or more users to access the health profile associated with the one or more affected users, one or more access to the health record will under the strict vigil eye to the one or more health profile associated with the one or more users. In one embodiment, the method (220) may include recording the one or more access by the system (20) with a date, a time, an identity associated with the person accessing and the like. In one embodiment, the one or more users' self-access may be recorded as well, for safety and security of the one or more details associated with the one or more users.

Further, the method (220) includes determining, by an emergency determining subsystem, one or more emergencies associated with the one or more affected users registered by the registration subsystem in step 240. In one embodiment, determining the one or more emergencies may include determining a road traffic accident, a referral, and the like. In one embodiment, the method (220) may include enabling, by the emergency determining subsystem, the one or more entities to upload a picture of the one or more affected users to determine at least one emergency from the one or more emergencies. In one embodiment, enabling, by an emergency panic subsystem, the one or more affected users to get the healthcare service without getting registered by the registration subsystem in case of an emergency. In one embodiment, the method (220) may include confirming, by the emergency panic subsystem, one or more affected users via a one-time password (OTP) for verification of the one or more affected users. Further, in one embodiment, the method (220) may include providing, by the emergency determining subsystem, end to end facilities, wherein the end to end facilities refers to intimate one or more current status from the start to the end of the treatment up to the discharge or the final outcome of the treatment associated with the one or more affected users will be updated continuously.

Further, in one embodiment, the method (220) may include providing, by the referral, a referral system from one or more rural areas (a primary health centre) to a secondary health centre and from the secondary health centre to a tertiary health centre for the treatment of the one or more affected users. In one embodiment, providing, by the referral, one or more referred affected users from one health centre to a higher health centre an ambulance service, as well as a prior emergency appointment, will be fixed with a higher health centre. In one embodiment, the method (220) may include sharing, by the referral system, a current health condition and reason for a refer and copy of a referral card associated with the one or more affected users with the higher health centre so that as soon as the one or more affected users reaches to a referral hospital the one or more affected users is attended instantly to get started treatment with provisional diagnosis shared earlier via health record. In one embodiment, notifying, by the referral system, the higher health centre about the estimated arrival time of the one or more affected users with current condition that the referral hospital can arrange the facilities accordingly.

Further, the method (220) includes enabling, by a healthcare service selection subsystem, the one or more affected users to select one or more healthcare services based on the one or more emergencies determined by the emergency determining subsystem in step 250. In one embodiment, selecting the one or more healthcare services may include selecting an ambulance service, a hospital service, a health worker service, a police service, a fire service and the like. In one embodiment, the method (220) may include generating, by an alert generation subsystem, an alert signal for the one or more healthcare service providers via the one or more modes upon selection of the one or more health care services.

In one embodiment, selecting, by the one or more affected users or one or more bystanders, at least one of the police service or the fire service or ambulance service from the one or more healthcare services corresponding to the one or more emergencies determined by the emergency determining subsystem. In one embodiment, the method (220) may include capturing, by the one or more affected users or one or more bystanders, one or more site details of the one or more affected users for sending the one or more site details to the at least one of the police service or the fire service or the ambulance service. In one embodiment, capturing the one or more site details may include capturing a mobile number of received call, a location of the accident, and the like. In one embodiment, the method (220) may include navigating, by the ambulance service or the police service or the fire service, a location of the one or more affected users using a global positioning system (GPS) upon receiving the one or more site details from the one or more affected users or the one or more bystanders.

Further, the method (220) includes connecting, by a healthcare service provider connecting subsystem, the one or more healthcare service providers to the one or more affected users based on the one or more healthcare services selected in the healthcare service selection subsystem in step 260. In one embodiment, the method (220) may include connecting, by the healthcare service provider connecting subsystem, at least one of the ambulance service, the police service or the fire service to one of the one or more affected users upon navigating a location of the one or more affected users using a GPS.

In one exemplary embodiment, the method (220) may include filling, by the ambulance service, the police service or the fire service, data associated with the one or more affected users and upload one or more pictures of the one or more affected users to connect to one or more near-by hospital services from the one or more healthcare services.

Further, the method (220) may include, generating, by a notification generation subsystem, one or more notification signals for the one or more healthcare service providers upon connecting the one or more healthcare service providers by the healthcare service provider connecting subsystem. In one embodiment, generating, by the notification generation subsystem, notification for the at least five emergency contact numbers, the hospital, the ward service, the nurse, the pharmacist, the doctor, the observation ward, and the like. In one embodiment, notifying, by the notification generation subsystem, the ward person from the one or more healthcare services receive the one or more affected users to hand over the one or more affected users to the hospital and notifying, by the notification about the estimated arrival time of the patient with a live GPS location of the one or more affected users with current condition, so that the hospital will be aware about the expected emergency and arrange the facilities accordingly.

Further, in one embodiment, notifying, by the notification generation subsystem, the nurse to enter one or more treatments given to the one or more affected users for updating the health record of the one or more affected users in the health card via a voice note dictation or a text dictation. In one embodiment, notifying, by the notification generation subsystem, the ward service about one or more updates associated with the one or more affected users. In one embodiment, notifying the ward service about one or more updates may include notifying the ward service about a transfer, a next duty, a duty completion, and the like.

In one embodiment, selecting, by the one or more affected users, a routine healthcare service from the one or more healthcare services. In one embodiment, selecting the routine healthcare service may include selecting an out-care patient department (OPD) or an in-care patient department (IPD). In one embodiment, selecting, by the one or more affected users, one or more facilities from the one or more healthcare services upon selecting the OPD or the IPD from the one or more healthcare services.

Further, in one embodiment, selecting the one or more facilities may include selecting a doctor, a specialist, an appointment time, a hospital, and the like. In one embodiment, the routine healthcare service may include generating a personalised daily report for the one or more affected users. In one embodiment, exceeding, by the one or more affected users, a predefined age may get a special facility when the age of the one or more affected users. Further, in one embodiment, the method (220) may include giving, by the system (20), special priority to one or more senior affected users based on the TRIAGE system. Further, in one embodiment, the method ( ) may include providing, a separate queue to facilitate the one or more senior affected users special care. In one embodiment, providing, by the TRIAGE system, one or more categories.

In one embodiment, providing the one or more categories may include providing an expectant category in black colour, an immediate category in red colour, a delayed category in yellow colour, a minor category in green colour and the like.

Further, the method (220) includes receiving, by a payment receiving subsystem, a payment from the one or more affected users via a payment card upon connecting the one or more healthcare service providers by the healthcare service provider subsystem in step 270. In one embodiment, the method (220) may include sharing, by the one or more affected users, the payment card with one or more family members of the one or more affected users upon receiving approval of the one or more affected users. In one embodiment, sharing with the payment card may include sharing with a credit card, a debit card, a cashless healthcare policy or a policy reimbursement and the like.

In one embodiment, connecting, by the payment receiving subsystem, one or more healthcare policy provider for payment if the one or more affected users includes a health care policy. Further, in one embodiment, the method (220) may include receiving, by the payment receiving subsystem, a discharge summary associated with the one or more associated users along with the payment. In another embodiment, the method (220) may include generating, by the payment receiving subsystem, an invoice and send to an insurance company associated with the one or more affected users to claim the health insurance.

Furthermore, in one specific embodiment, the method (220) may include analysing, by an analysing subsystem, working of one or more government health systems. In one embodiment analysing the one or more government health systems may include analysing a government health institution, a government health centre and the like. In one embodiment, analysing, by the analysing subsystem, negligence in the working of the one or more government health systems.

Various embodiments of the present disclosure provide a technical solution to the problem of providing accident and medical response. The present system provides an efficient system to provide healthcare services immediately to prevent fatal consequences which in turn reducing the amount of deaths and loss of economy. The present system also provides a secured system by providing multiple details about vehicle financial risks associated with the one or more affected users. The present disclosure also provides a health care facility to store previous health records of patients and identity proof of the corresponding patient which helps in analysing a health record of a patient for doctors as well as hospitals in a very easy manner and consumes less amount of time. Further, the present disclosure enables the user to make cashless payments through a payment card, a cashless healthcare policy or a policy reimbursement which results in immediate aid for the patient and therefore reduces chances of fatal consequences.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown; nor do all of the acts need to be necessarily performed. Also, those acts that are not dependant on other acts may be performed in parallel with the other acts. The scope of embodiments is by no means limited by these specific examples.

We claim:

1. A system (20) for accident and medical emergency response, the system (20) comprising:
one or more processors (30);
a registration subsystem (40) operable by the one or more processors (30), wherein the registration subsystem (40) is configured to register one or more entities, wherein the one or more entities comprise one or more affected users and one or more healthcare service providers;
an emergency determining subsystem (70) operable by the one or more processors (30), wherein the emergency determining subsystem (70) is configured to determine one or more emergencies associated with the one or more affected users registered by the registration subsystem (40);
a healthcare service selection subsystem (80) operable by the one or more processors (30), wherein the healthcare service selection subsystem (80) is configured to enable the one or more affected users to select one or more healthcare services based on the one or more emergencies determined by the emergency determining subsystem (70);
a healthcare service provider connecting subsystem (100) operable by the one or more processors (30), wherein the healthcare service provider connecting subsystem (100) is configured to connect the one or more healthcare service providers to the one or more affected users based on the one or more healthcare services selected in the healthcare service selection subsystem (80); and
a payment receiving subsystem (120) operable by the one or more processors (30), wherein the payment receiving subsystem (120) is configured to receive a payment from the one or more affected users via a payment card upon connecting the one or more healthcare service providers by the healthcare service provider connecting subsystem (100).

2. The system (20) as claimed in claim 1, wherein the one or more services comprise an ambulance service, a hospital service and a health worker service.

3. The system (20) as claimed in claim 1, wherein the payment card is shared within a family, as a family tree profile of the one or more affected users.

4. The system (20) as claimed in claim 1, comprising a health card generation subsystem configured to generate a health card comprising a health card registration number for the one or more affected users registered by the registration subsystem.

5. The system (20) as claimed in claim 1, comprising a notification generation subsystem configured to generate one or more notification signals for the one or more healthcare service providers upon connecting the one or more healthcare service providers by the healthcare service provider connecting subsystem.

6. The system (20) as claimed in claim 1, comprising a referral subsystem configured to refer the one or more affected users from one or more rural areas (a primary health centre) to a secondary health centre and from the secondary health centre to a tertiary health centre for the treatment.

7. A method (220) for accident and medical emergency response, the method (220) comprising:
registering, by a registration subsystem, one or more entities, wherein the one or more entities comprise one or more affected users and one or more healthcare service providers (230);
determining, by an emergency determining subsystem, one or more emergencies associated with the one or more affected users registered by the registration subsystem (240);
enabling, by a healthcare service selection subsystem, the one or more affected users to select one or more healthcare services based on the one or more emergencies determined by the emergency determining subsystem (250);
connecting, by a healthcare service provider connecting subsystem, the one or more healthcare service providers based on the one or more health care services selected by the one or more affected users in the healthcare service selection subsystem (260); and
receiving, by a payment receiving subsystem, a payment from the one or more affected users via a payment card upon connecting the one or more healthcare services by the healthcare service provider subsystem (270).

8. The method (220) as claimed in claim 7, wherein enabling the one or more affected users to select the one or more healthcare services comprise enabling the one or more affected users to select an ambulance service, a hospital service and a health worker service.

9. The method (220) as claimed in claim 7, wherein receiving the payment from the one or more affected users via the payment card comprises receiving the payment from the one or more affected users via the payment card shared within a family as a family tree profile of the one or more affected users.

10. The method (220) as claimed in claim 7, comprising generating, by a health card generation subsystem, a health card comprising a health card registration number for the one or more affected users registered by the registration subsystem.

11. The method (220) as claimed in claim 7, comprising generating, by a notification subsystem, one or more notification signals for the one or more healthcare service providers upon connection of the one or more healthcare service providers by the healthcare service provider connecting subsystem.

12. The method (220) as claimed in claim 7, comprising referring, by a referral subsystem, one or more affected users from one or more rural areas (a primary health centre) to a secondary health centre and from the secondary health centre to a tertiary health centre for the treatment.
